(19) United States
(12) Patent Application Publication
(10) Pub. No.: US 2009/0010826 A1
(43) Pub. Date: Jan. 8, 2009

(54) Toothbrush Sterilizer

(75) Inventor: Choong-Sik Shin, Seoul (KR)

Correspondence Address: RABIN & Berdo, PC, 1101 14TH STREET, NW, SUITE 500, WASHINGTON, DC, 20005, US

(73) Assignee: ESENCIA Co., Ltd., Seoul (KR)

(21) Appl. No.: 12/281,598

(22) PCT Filed: Mar. 2, 2007
(86) PCT No.: PCT/KR07/01059

§ 371 Date: Sep. 3, 2008

(30) Foreign Application Priority Data
Mar. 3, 2006 (KR) 10-2006-0020613

(51) Int. Cl. A61L 2/10

(52) U.S. Cl. 422/300

(57) ABSTRACT

A toothbrush sterilizer is provided. The toothbrush sterilizer employs a magnet on its pivoting cover and a lead switch on its main body opposite the cover to illuminate its sterilizing lamp only when the cover is closed, has a detachable toothbrush storage compartment, has a lamp holder that allows easy replacement of the sterilizing lamp and a detachably mounted stand with a water receiving area, and is easy to use and maintain in a hygienic state. The toothbrush sterilizer includes a storage compartment detachably attached inside the main body. The storage compartment has a plate, a side wall erected around a perimeter of the plate, and a plurality of insert holes formed in and extending a predetermined distance down from the plate, for storing the toothbrushes therein.

### TECHNICAL FIELD

The present invention relates to a toothbrush sterilizer, and more particularly, to a toothbrush sterilizer that employs a magnet on its pivoting cover and a lead switch on its main body opposite the cover to illuminate its sterilizing lamp only when the cover is closed, allows a compartment for storing toothbrushes to be detached from the main body, allows easy replacement of the sterilizing lamp due to a sterilizing lamp holder, has a detachably mounted stand forming a water receiving area at the bottom of the main body, and is easy to use and maintain in a hygienic state.

### BACKGROUND ART

A toothbrush sterilizer is a device that uses a sterilizing lamp that emits ultraviolet rays to sterilize toothbrush bristles, and includes a main body, holders for storing toothbrushes, and a sterilizing lamp.

Such toothbrush sterilizers according to the related art are divided into portable toothbrush sterilizers and standing units that are furnished and used in a bathroom.

Standing toothbrush sterilizers contain toothbrush holders inside the main body for storing a plurality of toothbrushes. A sterilizing lamp that emits ultraviolet rays onto toothbrush bristles and a controller for the lamp are provided behind the toothbrush holders. A cover that pivots on a hinge is provided at the front of the main body.

Furthermore, a mounting bracket that may be fastened to a wall may be included to mount the rear or lower end of the main body on, or a base may be included on which the main body may be placed upright.

Such a toothbrush sterilizer of the related art includes a switch for controlling the sterilizing lamp on a side of the main body. Such a switch is usually a sliding or a limit switch.

A sliding switch protrudes outward from a side of the main body and switches power on and off depending on its position. However, due to the inconvenience caused by a user having to manually turn the switch on and off, a limit switch has recently found favor.

A limit switch is provided in a location where the main body and the cover come together, so that when the cover closes the main body, the limit switch is pressed to activate the sterilizing lamp.

However, in the above configuration, the limit switch may be pressed manually when the cover is open, so that the sterilizing lamp is activated and ultraviolet rays are emitted to the outside of the unit, which may cause burns.

Also, the sterilizing lamp provided inside the main body is coupled to a socket of the main body. When the sterilizing lamp is detached from the socket to replace the lamp, the coupling portion can be easily damaged.

Especially in the case of vertically disposed sterilizing lamps, the lower portion of the lamp is fixed to the socket, so that even small shocks or vibrations cause severe movement of the lamp. Consequently, there is the possibility that the lamp may collide with the main body and be damaged, or that an electrode pin inserted into the socket may be severed.

Additionally, the toothbrush holders that hold toothbrushes are usually made of a plurality of elastic arms. The toothbrushes are inserted between the elastic arms. However, because the toothbrush bristles rest on the top surfaces of the elastic arms, the regions on the elastic arms that contact the bristles discolor, and the contaminants thereon may be transferred to the bristles.

Thus, a user is inconvenienced by having to frequently clean the toothbrush holders.

Furthermore, a separate water pan is not provided at the bottom of the main body, so that when water drains down from toothbrushes held in the holders, the water contaminates the floor of the unit.

### DISCLOSURE OF INVENTION

### Technical Problem

An object of the present invention is to provide a toothbrush sterilizer that employs a magnet on its pivoting cover and a lead switch on its main body opposite the cover to illuminate its sterilizing lamp only when the cover is closed. Because the switch does not protrude out from the main body, the turning on of the switch manually and ultraviolet rays emitted to the outside as a result can be prevented.

Another object of the present invention is to provide a toothbrush sterilizer with a fixing holder at the bottom of the sterilizing lamp. The lamp is inserted through the fixing holder into the socket, and the holder is fixed through protrusions so that the fastening force of the sterilizing lamp is increased. Thus, attaching and detaching the lamp for replacement is easier.

A further object of the present invention is to provide a toothbrush sterilizer with a plurality of toothbrush holders that are detachable from the main body and a reflective plate formed around the sterilizing lamp. The toothbrush holders hold the toothbrushes upright and lack a surface contacting the bristles, to prevent contamination of the bristles or discoloring of the holders. The reflective plate reflects and widens the range covered by the ultraviolet rays from the sterilizing lamp, so that sterilizing effectiveness of the unit increases.

A still further object of the present invention is to provide a toothbrush sterilizer with a fan at the rear of the main body. The fan operates to vent air into the main body, so that when a dryer and the sterilizing lamp radiate heat, the sterilizing capacity increases, and the sterilizing lamp can be operated for a long duration.

An even further object of the present invention is to provide a toothbrush sterilizer that has a pin protruding from the bottom of the main body for pressing the limit switch. In this way, a double safety mechanism is provided with this safety switch that senses whether the main body is securely placed on a floor, in order to prevent manual activation of the sterilizing lamp.

An additional object of the present invention is to provide a toothbrush sterilizer that has a detachable stand forming a water pan at the bottom of the main body, in order to prevent random dispersion of water that drains from toothbrushes stored in the main body onto the floor. Because the stand holds the water, contamination of the main body floor can be prevented.

### Technical Solution

According to an aspect of the present invention, there is provided a toothbrush sterilizer including: a main body for storing toothbrushes therein and having an open portion, an ultraviolet ray emitter provided within the main body for sterilizing the toothbrushes, and a pivoting cover for opening and closing the open portion, wherein the toothbrush sterilizer includes: a storage compartment detachably attached inside the main body, the storage compartment including: a plate; a side wall erected around a perimeter of the plate; and a plurality of insert holes formed in and extending a pre-determined distance down from the plate, for storing the toothbrushes therein.

The storage compartment may further include a plurality of electric toothbrush holes for storing heads of electric toothbrushes, and the storage compartment may further include an extending tube extending downward from the plate for forming the insert hole in a middle thereof, and a packing detachably and selectively formed on the extending tube, the packing having a through hole formed therein with an inner diameter smaller than a diameter of the insert hole.

The storage compartment may further include: a storage hole formed in the plate at a rear side wall for inserting a sterilizing lamp in, a guiding member having a guide slot formed in either side thereof, and a plurality of second air passage holes formed in the rear side wall, wherein the guiding member has a latch hole formed at a lower portion thereof and an elastic member with cut portions on either side of the guiding member; and a reflective plate inserted into the guiding member, being recessed in a semicircular shape for accommodating the sterilizing lamp in a middle thereof, and having a guide protrusion formed on either side at a rear thereof and a catch formed at a rear lower portion thereof for latching into the latch hole of the elastic member.

The reflective plate may further have a supporting holder formed at an inner, upper portion thereof, and the cover may have a magnet formed at a side thereof, and the main body may have a lead switch formed at a location thereon that corresponds to a location of the magnet when the cover is closed.

The main body may further have: a socket with a plurality of protrusions formed on edges thereof, the socket for inserting the sterilizing lamp in; and a holder forming a plurality of protrusion holes for inserting the protrusions, electrode pins formed at a bottom thereof for inserting into the socket, and the sterilizing lamp fixed thereabove.

The toothbrush sterilizer may further include a stand formed below the main body for maintaining a level height thereof, wherein the stand may include a first stand fixed to a lower rear portion of the main body and a second stand detachably attached at a front of the first stand and having a water receiving area recessed downward from an upper surface thereof.

The toothbrush sterilizer may further include: a limit switch on the ultraviolet ray emitter; and a protruding pin formed to protrude downward from a lower end of the main body for pressing the limit switch, wherein the protruding pin presses the limit switch for activating the ultraviolet ray emitter only when the main body is securely supported.

The main body may further have a supporting member, and the cover may have a pivoting member supported by the supporting member, the supporting member and the pivoting member allowing an opening and closing of the cover with respect to the main body, wherein the pivoting member may include a first shaft having one end thereof fixed to one side of the supporting member and the other end formed in a chevron, a second shaft having one end formed in a chevron and disposed opposite to the chevron of the first shaft and the other end extending to slide in the other side of the supporting member, and a spring disposed between the second shaft and the supporting member for providing elasticity to the second shaft.

The toothbrush sterilizer may further include a fan at a region of the main body.

The toothbrush sterilizer may further include: a left and a right side wall of the main body including a second latch hole formed respectively in a lower portion thereof; and a toothpaste compartment having a second hook on a side thereof for hooking in the second latch hole, wherein the toothpaste compartment is attached to one of the left and the right side walls.

### ADVANTAGEOUS EFFECTS

The toothbrush sterilizer of the present invention employs a magnet on its pivoting cover and a lead switch on its main body opposite the cover to illuminate its sterilizing lamp only when the cover is closed. Because the switch does not protrude out from the main body, the turning on of the switch manually and ultraviolet rays emitted to the outside as a result can be prevented.

The toothbrush sterilizer has a fixing holder at the bottom of the sterilizing lamp. The lamp is inserted through the fixing holder into the socket, and the holder is fixed through protrusions so that the fastening force of the sterilizing lamp is increased. Thus, attaching and detaching the lamp for replacement is easier.

The toothbrush sterilizer has a plurality of toothbrush holders that are detachable from the main body and a reflective plate formed around the sterilizing lamp. The toothbrush holders hold the toothbrushes upright and lack a surface contacting the bristles, to prevent contamination of the bristles or discoloring of the holders. The reflective plate reflects and widens the range covered by the ultraviolet rays from the sterilizing lamp, so that sterilizing effectiveness of the unit increases.

The toothbrush sterilizer has a fan at the rear of the main body. The fan operates to vent air into the main body, so that when a dryer and the sterilizing lamp radiate heat, the sterilizing capacity increases, and the sterilizing lamp can be operated for a long duration.

The toothbrush sterilizer has a pin protruding from the bottom of the main body for pressing the limit switch. In this way, a double safety mechanism is provided with this safety switch that senses whether the main body is securely placed on a floor, in order to prevent manual activation of the sterilizing lamp.

The toothbrush sterilizer has a detachable stand forming a water pan at the bottom of the main body, in order to prevent random dispersion of water that drains from toothbrushes stored in the main body onto the floor. Because the stand holds the water, contamination of the main body floor can be prevented.

### BRIEF DESCRIPTION OF THE DRAWINGS

The accompanying drawings, which are included to provide a further understanding of the invention and are incorporated in and constitute a part of this application, illustrate embodiment(s) of the invention and together with the description serve to explain the principle of the invention. In the drawings:

FIG. 1 is an exploded perspective view of a toothbrush sterilizer according to the present invention;

FIG. 2 is a perspective view of a toothbrush sterilizer with a pivoting cover according to the present invention;

FIG. 3 is a sectional view of a toothbrush sterilizer with its cover closed according to the present invention;

FIG. 4 is a frontal perspective view showing a main body detached from a second stand according to the present invention;

FIG. 5 is an enlarged sectional view showing the lower portion of a main body detached from a second stand and a mounting bracket according to the present invention;

FIG. 6 is a sectional view of FIG. 5 in an assembled state;

FIG. 7 is a partial exploded perspective view showing a storage compartment disassembled from a main body of a toothbrush sterilizer according to the present invention;

FIG. 8 is a partial sectional view showing the storage compartment disassembled from the main body in FIG. 7;

FIG. 9 is a partial, enlarged sectional view showing an assembled main body and storage compartment of a toothbrush sterilizer according to the present invention;

FIG. 10 is an enlarged sectional view showing a reflective plate disassembled from a storage compartment of a toothbrush sterilizer according to the present invention;

FIG. 11 is a partial, enlarged sectional view showing a cap-type hinge structure for a cover of a toothbrush sterilizer according to the present invention, where the cap is in its lowest position;

FIG. 12 is a partial, enlarged sectional view showing the cap in FIG. 11 in its highest position; and

FIG. 13 is a perspective view of a toothbrush sterilizer with its cover open, according to another embodiment of the present invention.

### BEST MODE FOR CARRYING OUT THE INVENTION

Reference will now be made in detail to the preferred embodiments of the present invention, examples of which are illustrated in the accompanying drawings. Wherever possible, the same reference numbers will be used throughout the drawings to refer to the same or like parts.

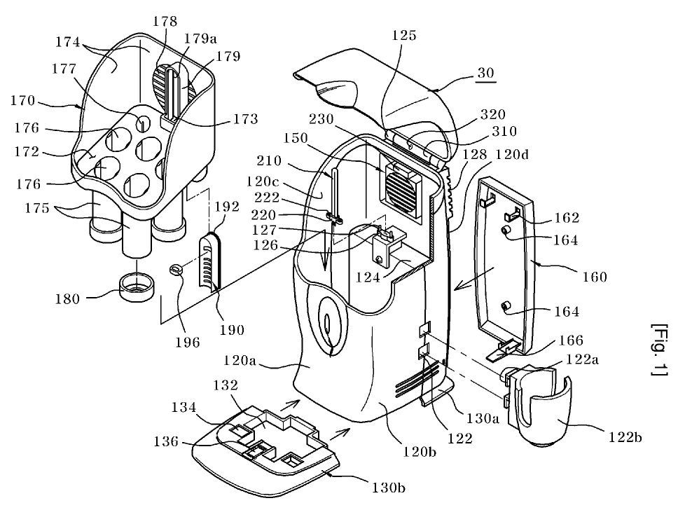

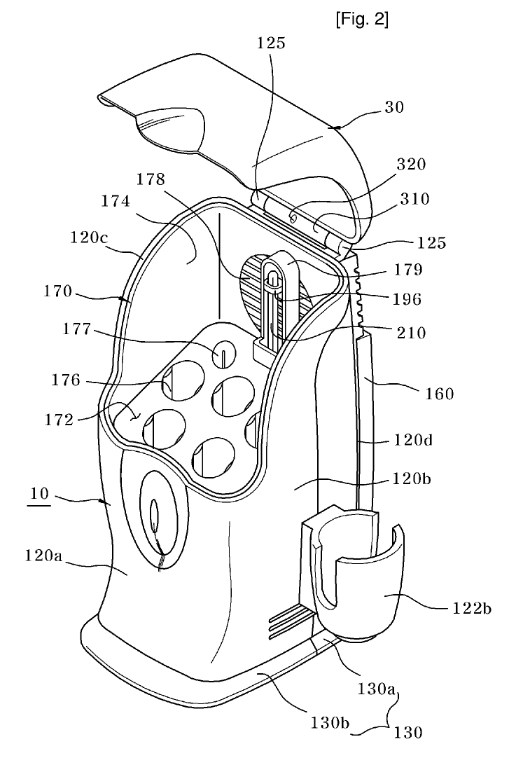

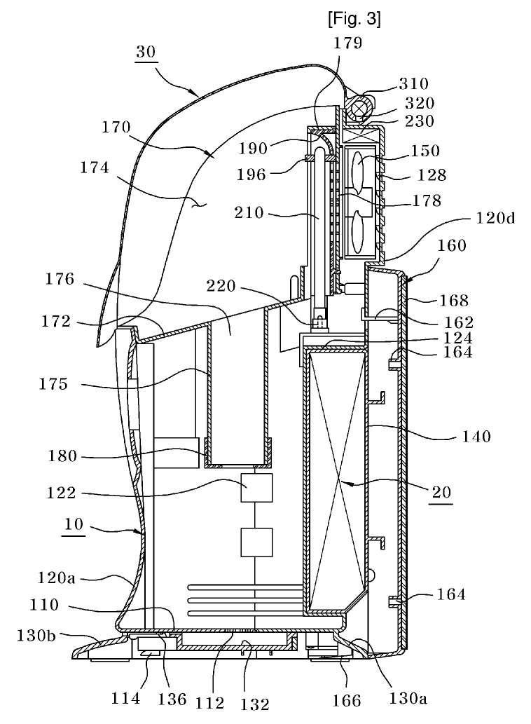

FIG. 1 is an exploded perspective view of a toothbrush sterilizer according to the present invention, FIG. 2 is a perspective view of a toothbrush sterilizer with a pivoting cover according to the present invention, and FIG. 3 is a sectional view of a toothbrush sterilizer with its cover closed according to the present invention. Referring to FIGS. 1 through 3, a toothbrush sterilizer is open at one side and includes a main body 10 for storing toothbrushes, an ultraviolet ray emitter 20 provided in the main body 10 for emitting ultraviolet rays, and a cover 30 for closing the open portion of the main body 10.

In further detail, the main body 10 includes a bottom surface 110 and side walls 120a, 120b, 120c, and 120d rising from the floor to form a storage space within. The main body 10 is formed with two vertical halves a coupled structure that is well known to those with general knowledge of the art—and thus, a description of the structure will be omitted.

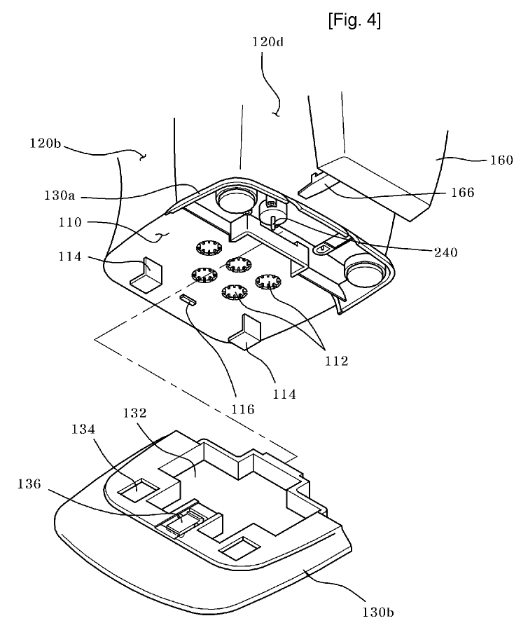

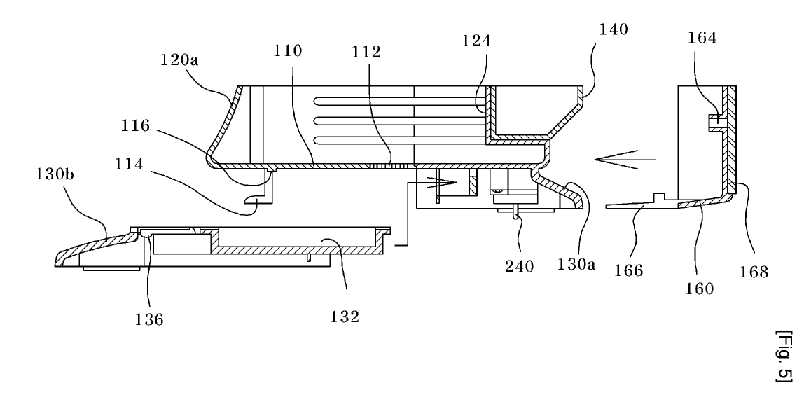

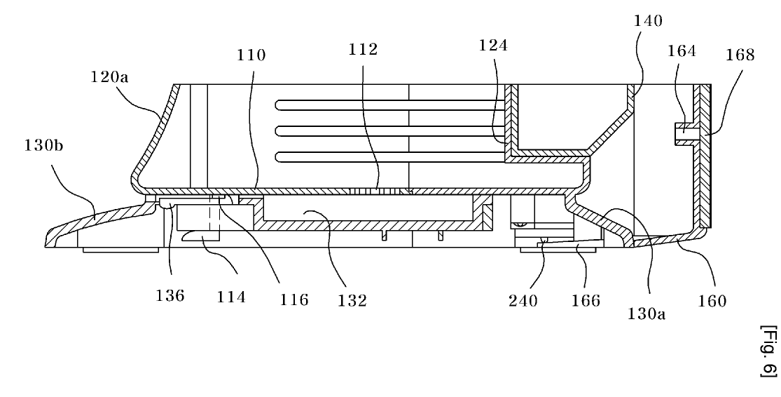

FIG. 4 is a frontal perspective view showing a main body detached from a second stand according to the present invention, FIG. 5 is an enlarged sectional view showing the lower portion of a main body detached from a second stand and a mounting bracket according to the present invention, and FIG. 6 is a sectional view of FIG. 5 in an assembled state. Referring to FIGS. 4 through 6, the bottom surface 110 defines a plurality of drain holes 112, and a stand is formed to extend a predetermined distance downward from the bottom of the bottom surface 110. The stand includes a first stand 130a fixed below the rear of the floor and a second stand 130b detachably connected at the front of the first stand.

The second stand 130b has a water pan recessed a predetermined depth to collect water that drains through the drain holes 112.

Moreover, the second stand 130b has a detachable structure that includes a plurality of first latch holes 134 formed through an upper surface of the second stand 130b, and an elastic catch 136 formed on the top, front surface thereof, with an elastic front portion and a hook at the rear. Corresponding to the first latch holes 134 and elastic catch 136 are first hooks 114 and a catch protrusion 116 formed on the bottom of the bottom surface 110.

Also, a guide recess may be formed in the top surface of the second stand 130b, and a guide protrusion may be formed to protrude from the bottom of the bottom surface 110 to be guided by the guide recess.

A display for displaying the operation of the sterilizing lamp and the fan is formed on the front of the side wall 120a.

A second latch hole 122 is formed respectively at the lower portions in the side walls 120b and 120c, for latching a second hook 122a formed on a side of a toothpaste compartment 122b.

When the toothpaste compartment 122b is hung and fixed on one of the side walls 120b and 120c, the second latch hole 122 on the remaining side wall may be sealed with a packing.

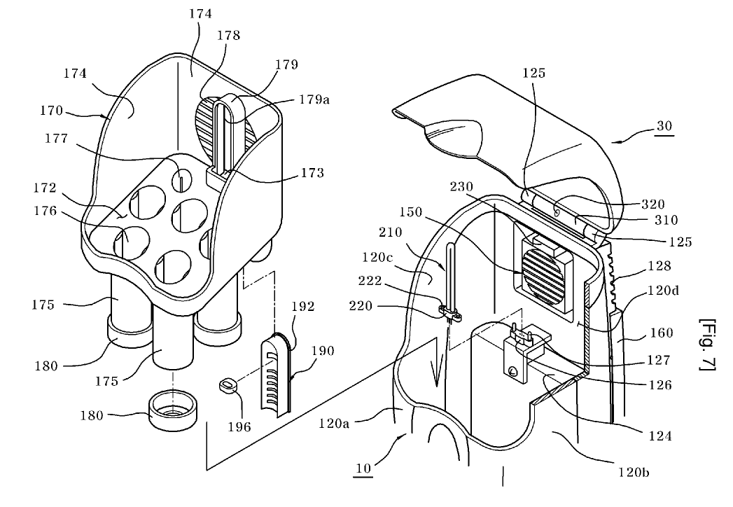

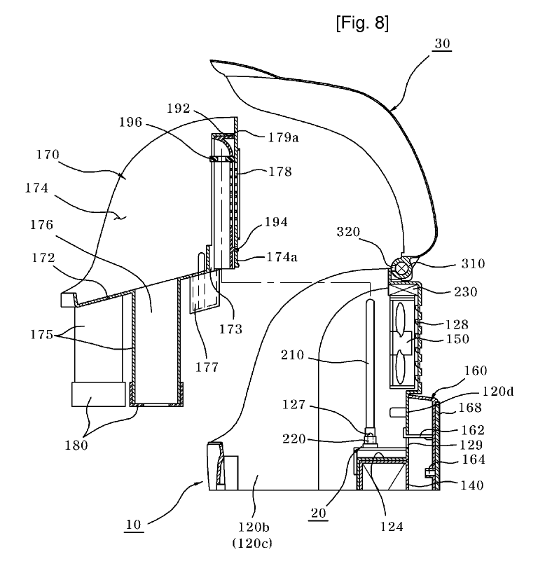

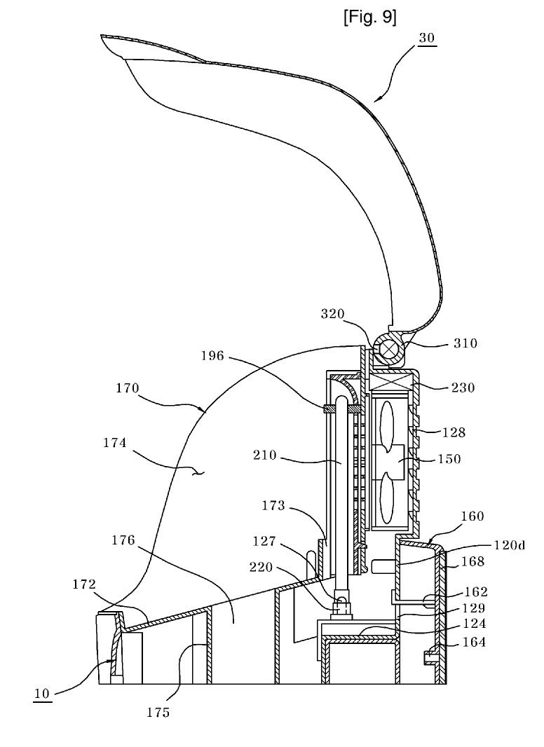

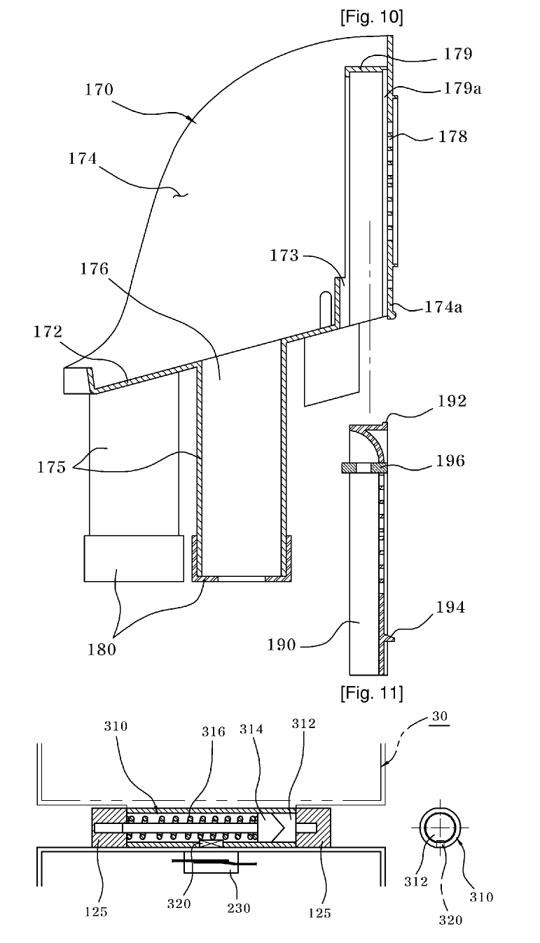

FIG. 7 is a partial exploded perspective view showing a storage compartment disassembled from a main body of a toothbrush sterilizer according to the present invention, FIG. 8 is a partial sectional view showing the storage compartment disassembled from the main body in FIG. 7, FIG. 9 is a partial, enlarged sectional view showing an assembled main body and storage compartment of a toothbrush sterilizer according to the present invention, and FIG. 10 is an enlarged sectional view showing a reflective plate disassembled from a storage compartment of a toothbrush sterilizer according to the present invention. Referring to FIGS. 7 through 10, a stepped compartment 124 for storing a circuit board of the ultraviolet emitter 20 is recessed in the side wall 120d to have a predetermined height. The amount that it is recessed determines its inner space, and a finishing panel 140 is mounted at the rear of the side wall 120d to cover the space.

Additionally, a socket 126, connected to the circuit board for the sterilizing lamp to insert into, is formed on top of the stepped compartment 124 of the side wall 120d. The socket has a protrusion 127 formed on either side thereof.

A fan 150 is fixed within the upper portion of the side wall 120d, and the region of the side wall 120d where the fan is located has a plurality of first air passage holes 128 formed therein. The fan 150 may be provided behind the space where the sterilizing lamp is erected.

A mounting bracket 160 is detachably fixed to the rear of the side wall 120d, and the mounting bracket 160 has a plurality of third hooks 162 protruding forward from the mounting bracket 160, a plurality of fastening holes 164 passing therethrough, and a supporting plate 166 protruding forward therefrom and having double-sided tape 168 on its bottom surface.

A third latch hole 129 is formed at the rear of the side wall 120d for inserting and fastening the third hook 162 of the mounting bracket 160 therein.

A supporting member 125, for supporting both ends of the pivoting member on the cover 30, is formed on the upper rear portion of the side wall 120d.

Referring to FIGS. 7 through 10, a storage compartment 170 for storing a plurality of toothbrushes is detachably disposed in the interior storage space of the main body 10.

The storage compartment 170 includes a plate 172 with a side wall 174 formed around its perimeter. The plate 172 has a plurality of insert holes 176 for inserting regular toothbrushes in and electric toothbrush holes 177 with protruding plates formed in the middle for supporting shorter electric toothbrush heads. A mix of a plurality of insert holes 176 and electric toothbrush holes 177 may be formed.

When the storage compartment 170 is inserted inside the main body 10, it may be supported by supporting elements (not shown) formed in plurality inside the main body 10.

Here, the installed height of the storage compartment 170 may be formed to allow toothbrushes that are stored in the insert holes 176 to be supported by the floor 110 and prevent the bristles from touching the plate 172.

In order to secure the storage compartment 170, a fastening screw may be screwed through the rear of the side wall 120d of the main body 10 into the storage compartment to fasten the latter.

An extending tube 175 is further formed to extend downward from the plate 172, so that the insert hole 176 communicates with the center of the extending tube 175. The plate 172 may be inclined at a predetermined angle.

The extending tube 175 has a packing 180 inserted over the bottom thereof, with a hole (smaller in diameter than the inner diameter of the extending tube 175 and that of the insert hole 176) formed through the packing 180. The packing may selectively be used to store childrens toothbrushes and toothbrushes that are short, preventing them from falling into the insert hole 176.

The packing 180 may be formed of a soft silicon or rubber material.

A plurality of second air passage holes 178 are formed in the side wall 174 at the rear of the storage compartment 170, and a storage hole 173 is formed in the plate 172 for the sterilizing lamp to pass through. A guide slot 179 is formed on either side of guiding member 179 that passes through the storage hole 173.

A reflective plate 190 has a guide protrusion 192 that is guided in the guide slot 179, a front that is concaved inward, and a catch 194 formed at the rear thereof.

In this case, an elastic member 174a, being elastic in order to receive and fix the catch 194, is formed on the side wall 174 facing the catch 194.

A supporting holder 196 is fixed at the upper, inner portion of the reflective plate 190, in order to support the upper portion of the sterilizing lamp when the latter is positioned at the front of the reflective plate 190.

Instead of using a separate supporting holder 196, the front of the reflective plate 190 may be partially cut, and the cut portion may be curved to form a space within.

Also, the reflective plate 190 may be coated with a reflective material on only its front concave portion or in its entirety, so that ultraviolet light emitted by the sterilizing lamp can be increased in range.

The ultraviolet ray emitter 20 includes a sterilizing lamp 210, a switch, a circuit board for controlling the former, and a power supply.

A holder 220 for supporting the lower end of the sterilizing lamp 210 is provided. The holder 220 has a protrusion hole 222 on either side thereof through which a protrusion 127 on the socket 126 inserts through, and electrode pins at the bottom. The switch may be a lead switch 230 that transmits an on/off signal according to its magnetization, and may be altered by the position of a magnet provided on a portion of the cover 30.

In this case, the lead switch 230 may be located at the top of the side wall 120d at the rear of the main body 10 or at the top of the side wall 120a at the front of the main body 10.

Besides the lead switch 230 that is a first safety measure, a limit switch (not shown), that is a second safety measure, may be employed.

The limit switch (not shown) is located at the bottom of the circuit board, and a protruding pin 240, that presses the limit switch, may protrude from the bottom of the floor 110 of the main body 10 further than the stand.

When the main body 10 is set upright on a level surface, the protruding pin 240 is pressed by the surface and presses the limit switch (not shown) to transmit an ‘on’ signal. Should the main body 10 fall over, the protruding pin 240 reassumes a protruding position to transmit an ‘off’ signal, so that the sterilizing lamp 210 and the fan 150 do not operate, despite the lead switch 230 being in an ‘on’ position.

When the main body 10 is mounted on the mounting bracket 160, the protruding pin 240 is pressed by the supporting plate 166 at the bottom of the mounting bracket 160.

In order to open and close the upper portion of the main body 10, the pivoting member 310 supported by the supporting member 125 is formed on an end of the cover 30, and the pivoting member 310 may be a cap-type structure that automatically opens the cover 30 when the cover 30 is opened beyond a certain angle.

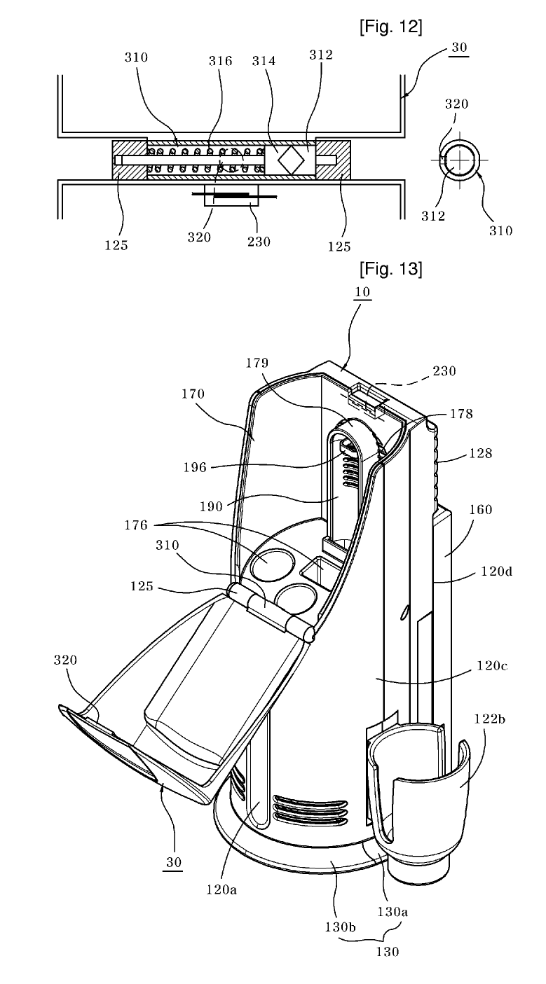

FIG. 11 is a partial, enlarged sectional view showing a cap-type hinge structure for a cover of a toothbrush sterilizer according to the present invention, where the cap is in its lowest position, and FIG. 12 is a partial, enlarged sectional view showing the cap in FIG. 11 in its highest position. Referring to FIGS. 11 and 12, the cap-type structure includes a first cap shaft 312 fixed at one end to a side of the supporting member 125 and having a chevron-shaped cap forming the other end; and a second cap shaft 314 having a chevron-shaped cap on one end thereof facing the cap of the first cap shaft 312, whereby the second cap shaft 314 extends to pivot within the opposite end of the supporting member 125. A spring 316 is provided between the second cap shaft 314 and the supporting member 125, to impart elasticity to the second cap shaft 314.

That is, the highest point of the first cap shaft 312 interlocks with the lowest point of the second cap shaft 314, and when the second cap shaft 314 is rotated, the highest points of both caps come into mutual contact, so that the second cap shaft 314 is designed to slide within its inserted space in the supporting member 125.

The first cap shaft 312 is fixed in the supporting member 125, regardless of the opening movement of the cover 30. However, the second cap shaft 314 may be inserted to pivot in concert with the pivoting of the cover 30.

Additionally, the magnet 320 may be positioned to magnetize the lead switch 230 when the cover 30 is closed and may be provided on an area of the pivoting member 310 or on the end of the cover across from the pivoting member 310, as shown in FIG. 13.

A description of the assembly of the above-structured toothbrush sterilizer will now be given.

The fan 150 is inserted in the rear side wall 120d of the main body 10, and the sterilizing lamp 210, inserted in the socket 126 and including the holder 220 fixed on the protrusions 127, is located in front of the fan 150.

The storage compartment 170 is provided within the main body 10, and the catch 194 of the reflective plate 190 inserted into the guiding member 179 is received and fixed in the elastic member 174a, and the sterilizing lamp 210 is inserted through the storage hole 173. The reflective plate 190 is located at the rear of the sterilizing lamp 210.

The upper portion of the sterilizing lamp 210 is inserted and supported in the space formed by the supporting holder 196 formed on the reflective plate 190.

The toothbrush sterilizer configured as above is used in the following manner.

First, depending on where the toothbrush sterilizer is to be located, it is either set upright on a flat surface with the mounting bracket 160 detached, or the mounting bracket 160 is attached to a wall.

Here, the mounting bracket 160 is fixed to a wall using double-sided tape 168 or screws or nails driven through a fastening hole 164. The third hook 162 protruding forward from the mounting bracket 160 is inserted in the third latch hole 129 of the main body 10, so that the main body maintains a hooked position on the mounting bracket 160. The supporting plate 166 extending from the bottom of the mounting bracket 160 presses the protruding pin 240. Below, a thus hanging main body 10 will be described.

The supporting plate 166 pressing the protruding pin 240, in turn, presses the limit switch (not shown) that transmits an on signal to the circuit of the ultraviolet emitter 20.

Then, the cover 30 is pivoted and opens automatically after it is opened past a certain angle, due to the cap-type pivoting member 310.

In further detail, the second cap shaft 314 (which is pressed against the head of the first cap shaft 312 pivots an angle corresponding to the angle that the cover 30 is opened, so that when the heads of the first and second cap shafts 312 and 314 reach their highest points against each other, the spring 316 exerts its maximum pressure and exerts a returning force. When an angle past the highest point is reached, the returning force of the spring 316 pressure is applied to the second cap shaft 314 to automatically return it to its lowest point through rotation.

Accordingly, the cover 30 that pivots in concert with the second cap shaft 314 opens completely.

In the above main body 10, when the cover 30 opens completely, toothbrushes stored in the insert holes 176 of the storage compartment 170 may be removed and used. After use, the toothbrushes are replaced in the insert holes 176.

Then, the lower ends of the toothbrushes stored in the insert holes 176 of the storage compartment 170 contact the floor 110. Here, the toothbrushes are stored upright, and their bristles do not come into contact with the plate 172 of the storage compartment 170.

After the toothbrushes are placed in the storage compartment 170, the opened cover 30 is pivoted in the opposite direction to close the upper portion of the main body 10. Here, the pivoting member 310 automatically closes the cover 30 in a reverse process of that described above after the cover 30 is pivoted beyond a certain angle.

The magnet 320 is provided on the pivoting member 310 of the closing cover 30, so that when the cover 30 is closed, the magnet 320 is disposed at bottom position. Because the limit switch 230 is coupled on the main body 10 at a location below the magnet 320, the magnetism of the magnet 320 causes the lead switch 230 to transmit an on signal.

When both the lead switch 230 and the limit switch (not shown), that is pressed by the protruding pin 240 that is pressed by the supporting plate 166 of the mounting bracket 160, transmit on signals, the circuit board applies power to the sterilizing lamp 210 and the fan 150 to activate them.

Therefore, the ultraviolet rays emitted from the sterilizing lamp 210 sterilize the toothbrushes, and the airflow from the fan 150 quickly dries the bristles to increase sterilizing effectiveness. Also, the reflective plate 190 provided behind the sterilizing lamp 210 magnifies and reflects the ultraviolet rays for even further sterilizing effectiveness.

The sterilizing lamp 210 and the fan 150 may be made to operate for a preset duration.

Moisture from the toothbrushes stored in the storage compartment 170 flows down through the drain holes 112 formed in the floor 110 and is collected in the water receiving area 132 of the second stand 130b. In this way, moisture dripping down to contaminate other parts of the apparatus in the related art can be prevented.

When the storage compartment 170 of the toothbrush sterilizer requires cleaning after prolonged use, the main body 10 may be detached from the mounting bracket 160, and the storage compartment 170 may be disassembled after unscrewing a fastening screw fixing the storage compartment 170 to the main body 10 at the rear side wall 120d.

Subsequently, the detached storage compartment 170 can be easily cleaned. Also, the packings 180 may be inserted over the lower ends of the extending tubes 175 to allow storage of childrens and similar small-sized toothbrushes.

Thus, the toothbrush sterilizer uses the lead switch to accurately determine if the cover is closed to prevent manually-induced emitting of ultraviolet rays to the outside, and the limit switch as a second safety measure to prevent ultraviolet ray emitting to the outside and minimize power consumption.

Additionally, because the storage compartment is removable from the main body, a clean state can be maintained, and discoloration of the unit due to toothbrush bristles coming into contact with its surfaces can be prevented. Also, sterilizing effectiveness is increased by using the reflective plate that increases emitted ultraviolet rays.

### MODE FOR THE INVENTION

FIG. 13 is a perspective view of a toothbrush sterilizer with its cover open, according to another embodiment of the present invention. Referring to FIG. 13, a supporting member 125 for supporting a pivoting member 310 is formed at the front side wall of the main body 10, and a lead switch 230 is provided at the rear.

Also, in this configuration the pivoting member 310 of the cover 30 and the supporting member 125 supporting the pivoting member 310 are formed at the front lower portion of the main body 10. The magnet 320 is installed at the rear end of the cover 30, in order to sense when the cover 30 is opened and closed.

It will be apparent to those skilled in the art that various modifications and variations can be made in the toothbrush sterilizer according to the present invention. Thus, it is intended that the present invention covers the modifications and variations of this invention provided they come within the scope of the appended claims and their equivalents.

1. A toothbrush sterilizer having a main body (10) for storing toothbrushes therein and having an open portion, an ultraviolet ray emitter (20) provided within the main body (10) for sterilizing the toothbrushes, and a pivoting cover (30) for opening and closing the open portion, wherein the toothbrush sterilizer comprises:
a storage compartment (170) detachably attached inside the main body (10), the storage compartment including:
a plate (172);
a side wall (174) erected around a perimeter of the plate (172); and
a plurality of insert holes (176) formed in and extending a predetermined distance down from the plate, for storing the toothbrushes therein.

2. The toothbrush sterilizer of claim 1, wherein the storage compartment (170) further includes a plurality of electric toothbrush holes (177) for storing heads of electric toothbrushes.

3. The toothbrush sterilizer of claim 1, wherein the storage compartment (170) further includes an extending tube extending downward from the plate (172) for forming the insert hole (176) in a middle thereof, and a packing (180) detachably and selectively formed on the extending tube (175), the packing (180) having a through hole formed therein with an inner diameter smaller than a diameter of the insert hole (176).

4. The toothbrush sterilizer of claim 1, wherein the storage compartment (170) further includes:
a storage hole (173) formed in the plate at a rear side wall (174) for inserting a sterilizing lamp (210) in, a guiding member (179) having a guide slot (179a) formed in either side thereof, and a plurality of second air passage holes (178) formed in the rear side wall, wherein the guiding member (179) has a latch hole formed at a lower portion thereof and an elastic member (174a) with cut portions on either side of the guiding member; and
a reflective plate (190) inserted into the guiding member (179), being recessed in a semicircular shape for accommodating the sterilizing lamp (210) in a middle thereof, and having a guide protrusion (192) formed on either side at a rear thereof and a catch (194) formed at a rear lower portion thereof for latching into the latch hole of the elastic member (174a).

5. The toothbrush sterilizer of claim 4, wherein the reflective plate (190) further includes a supporting holder (196) formed at an inner, upper portion thereof.

6. The toothbrush sterilizer of claim 1, wherein the cover (30) includes a magnet (320) formed at a side thereof, and the main body (10) includes a lead switch (230) formed at a location thereon that corresponds to a location of the magnet (320) when the cover (30) is closed.

7. The toothbrush sterilizer of claim 1, wherein the main body (10) further includes:
a socket (126) with a plurality of protrusions (127) formed on edges thereof, the socket (126) for inserting the sterilizing lamp (210) in; and
a holder (220) forming a plurality of protrusion holes (222) for inserting the protrusions (127), electrode pins formed at a bottom thereof for inserting into the socket (126), and the sterilizing lamp (210) fixed thereabove.

8. The toothbrush sterilizer of claim 1, further comprising a stand formed below the main body (10) for maintaining a level height thereof, wherein the stand includes a first stand (130a) fixed to a lower rear portion of the main body (10) and a second stand (130b) detachably attached at a front of the first stand (130a) and having a water receiving area (132) recessed downward from an upper surface thereof.

9. The toothbrush sterilizer of claim 1, further comprising:
a limit switch on the ultraviolet ray emitter (20); and
a protruding pin (240) formed to protrude downward from a lower end of the main body (10) for pressing the limit switch, wherein the protruding pin (240) presses the limit switch for activating the ultraviolet ray emitter (20) only when the main body (10) is securely supported.

10. The toothbrush sterilizer of claim 1, wherein the main body (10) further has a supporting member (250);
the cover (30) has a pivoting member (310) supported by the supporting member (250), the supporting member (250) and the pivoting member (310) allowing an opening and closing of the cover (30) with respect to the main body (10), the pivoting member (310) including a first shaft (312) having one end thereof fixed to one side of the supporting member (250) and the other end formed in a chevron, a second shaft (314) having one end formed in a chevron and disposed opposite to the chevron of the first shaft (312) and the other end extending to slide in the other side of the supporting member (250), and a spring (316) disposed between the second shaft (314) and the supporting member (250) for providing elasticity to the second shaft (314).

11. The toothbrush sterilizer of claim 1, further comprising a fan (150) at a region of the main body (10).

12. The toothbrush sterilizer of claim 1, further comprising:
a left and a right side wall (120b),(120c) of the main body (10) including a second latch hole (122) formed respectively in a lower portion thereof; and
a toothpaste compartment (122b) having a second hook (122a) on a side thereof for hooking in the second latch hole (122), wherein the toothpaste compartment (122b) is attached to one of the left and the right side walls (120b),(120c).

13. The toothbrush sterilizer of claim 6, further comprising:
a limit switch on the ultraviolet ray emitter (20); and
a protruding pin (240) formed to protrude downward from a lower end of the main body (10) for pressing the limit switch, wherein the protruding pin (240) presses the limit switch for activating the ultraviolet ray emitter (20) only when the main body (10) is securely supported.

14. The toothbrush sterilizer of claim 7, further comprising:
a limit switch on the ultraviolet ray emitter (20); and
a protruding pin (240) formed to protrude downward from a lower end of the main body (10) for pressing the limit switch, wherein the protruding pin (240) presses the limit switch for activating the ultraviolet ray emitter (20) only when the main body (10) is securely supported.

15. The toothbrush sterilizer of claim 8, further comprising:
a limit switch on the ultraviolet ray emitter (20); and
a protruding pin (240) formed to protrude downward from a lower end of the main body (10) for pressing the limit switch, wherein the protruding pin (240) presses the limit switch for activating the ultraviolet ray emitter (20) only when the main body (10) is securely supported.

16. The toothbrush sterilizer of claim 6, wherein the main body (10) further has a supporting member (250);
the cover (30) has a pivoting member (310) supported by the supporting member (250), the supporting member (250) and the pivoting member (310) allowing an opening and closing of the cover (30) with respect to the main body (10), the pivoting member (310) including a first shaft (312) having one end thereof fixed to one side of the supporting member (250) and the other end formed in a chevron, a second shaft (314) having one end formed in a chevron and disposed opposite to the chevron of the first shaft (312) and the other end extending to slide in the other side of the supporting member (250), and a spring (316) disposed between the second shaft (314) and the supporting member (250) for providing elasticity to the second shaft (314).

17. The toothbrush sterilizer of claim 7, wherein the main body (10) further has a supporting member (250);
the cover (30) has a pivoting member (310) supported by the supporting member (250), the supporting member (250) and the pivoting member (310) allowing an opening and closing of the cover (30) with respect to the main body (10), the pivoting member (310) including a first shaft (312) having one end thereof fixed to one side of the supporting member (250) and the other end formed in a chevron, a second shaft (314) having one end formed in a chevron and disposed opposite to the chevron of the first shaft (312) and the other end extending to slide in the other side of the supporting member (250), and a spring (316) disposed between the second shaft (314) and the supporting member (250) for providing elasticity to the second shaft (314).

18. The toothbrush sterilizer of claim 8, wherein the main body (10) further has a supporting member (250);
the cover (30) has a pivoting member (310) supported by the supporting member (250), the supporting member (250) and the pivoting member (310) allowing an opening and closing of the cover (30) with respect to the main body (10), the pivoting member (310) including a first shaft (312) having one end thereof fixed to one side of the supporting member (250) and the other end formed in a chevron, a second shaft (314) having one end formed in a chevron and disposed opposite to the chevron of the first shaft (312) and the other end extending to slide in the other side of the supporting member (250), and a spring (316) disposed between the second shaft (314) and the supporting member (250) for providing elasticity to the second shaft (314).

19. The toothbrush sterilizer of claim 6, further comprising a fan (150) at a region of the main body (10).

20. The toothbrush sterilizer of claim 7, further comprising a fan (150) at a region of the main body (10).

21. The toothbrush sterilizer of claim 8, further comprising a fan (150) at a region of the main body (10).

22. The toothbrush sterilizer of claim 6, further comprising:
a left and a right side wall (120b),(120c) of the main body (10) including a second latch hole (122) formed respectively in a lower portion thereof; and
a toothpaste compartment (122b) having a second hook (122a) on a side thereof for hooking in the second latch hole (122), wherein the toothpaste compartment (122b) is attached to one of the left and the right side walls (120b),(120c).

23. The toothbrush sterilizer of claim 7, further comprising:
a left and a right side wall (120b),(120c) of the main body (10) including a second latch hole (122) formed respectively in a lower portion thereof; and
a toothpaste compartment (122b) having a second hook (122a) on a side thereof for hooking in the second latch hole (122), wherein the toothpaste compartment (122b) is attached to one of the left and the right side walls (120b),(120c).

24. The toothbrush sterilizer of claim 8, further comprising:
a left and a right side wall (120b),(120c) of the main body (10) including a second latch hole (122) formed respectively in a lower portion thereof; and
a toothpaste compartment (122b) having a second hook (122a) on a side thereof for hooking in the second latch hole (122), wherein the toothpaste compartment (122b) is attached to one of the left and the right side walls (120b),(120c).
